(19) United States
(12) Patent Application Publication
(10) Pub. No.: US 2009/0054836 A1
(43) Pub. Date: Feb. 26, 2009

(54) Method and Apparatus for Treating Stenoses at Bifurcated Regions

(75) Inventor: Sinead O'Donnell, Ballybrit (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/841,143

(22) Filed: Aug. 20, 2007

(51) Int. Cl. A61M 25/10

(52) U.S. Cl. 604/101.01; 606/194; 623/1.11

(57) ABSTRACT

A bifurcated catheter includes a first catheter branch having a first distal portion and a second catheter branch having a second distal portion. The first and second distal portions are linked together by a pocket disposed on the first distal portion of the first branch catheter. The pocket includes a proximal opening, a distal opening, and a slit. The pocket is sized and shaped to receive the second distal portion. When the second distal portion is inserted into the pocket, the first and second catheter branches are linked together for delivery to the bifurcated region over a first guidewire. Upon delivery to the bifurcated region, a guidewire is advanced through the second catheter branch and into a branch vessel. As first and second catheter branches are advanced over the first and second guidewires, respectively, a force pulling the catheter branches apart pulls the second distal portion through the slit of the pocket, thereby releasing the first and second catheter branches from each other.

### FIELD OF THE INVENTION

The invention relates generally dilatation catheters, stents and grafts for dilating strictures or stenoses in the human body. More particularly, the invention relates to a balloon catheter, including a delivery system for a bifurcated endoluminal prosthesis, for treating a site or sites at or near a bifurcation of a body lumen.

### BACKGROUND OF THE INVENTION

The use of balloon catheters with or without stents to treat strictures, stenoses, or narrowings in various parts of the human body is well known in the prior art. Devices of numerous designs have been utilized for angioplasty, stents and grafts or combination stent/grafts. Varied catheter designs have been developed for the dilatation of stenoses and to deliver prostheses to treatment sites within the body lumen.

Devices developed specifically to address the problems that arise in the treatment of stenoses at or near the site of a bifurcation of a body lumen are known in the art. Examples of catheters for use in treating bifurcated lumens or delivery systems for bifurcated endoluminal prostheses are shown in U.S. Pat. No. 5,720,735 to Dorros, U.S. Pat. No. 5,669,924 to Shaknovich, U.S. Pat. No. 5,749,825 to Fischell, et al., and U.S. Pat. No. 5,718,724 to Goicoechea et al.

Various techniques have been used to deliver multiple prostheses in order to provide radial support to both a main blood vessel, for example, and contemporaneously to side branches of the blood vessel. Further, single bifurcated stents and grafts have been developed in order to treat such conditions at the site of a branch of a body lumen. A bifurcated stent and/or graft typically comprises a tubular body or trunk and two tubular legs. Examples of bifurcated stents are shown in U.S. Pat. No. 5,723,004 to Dereume et al., U.S. Pat. No. 4,994,071 to MacGregor, and European Pat. Application EP 0 804 907 A2 to Richter, et al.

Illustrative procedures involving balloon catheters include percutaneous transluminal angioplasy (PTA) and percutaneous transluminal coronary angioplasty (PTCA), which may be used to reduce arterial build-up such as caused by the accumulation of atherosclerotic plaque. These procedures involve passing a balloon catheter over a guide wire to a stenosis with the aid of a guide catheter. The guide wire extends from a remote incision to the site of the stenosis, and typically across the lesion. The balloon catheter is passed over the guide wire, and ultimately positioned across the lesion.

Once the balloon catheter is positioned appropriately across the lesion, (e.g., under fluoroscopic guidance), the balloon is inflated, which breaks the plaque of the stenosis and causes the arterial cross section to increase. Then the balloon is deflated and withdrawn over the guide wire into the guide catheter, and from the body of the patient.

In many cases, a stent or other prosthesis must be implanted to provide permanent support for the artery. When such a device is to be implanted, a balloon catheter which carries a stent on its balloon is deployed at the site of the stenosis. The balloon and accompanying prosthesis are positioned at the location of the stenosis, and the balloon is inflated to circumferentially expand and thereby implant the prosthesis. Thereafter, the balloon is deflated and the catheter and the guide wire are withdrawn from the patient.

Administering PTCA and/or implanting a stent at a bifurcation in a body lumen poses further challenges for the effective treatment of stenoses in the lumen. For example, dilating a vessel at a bifurcation may cause narrowing of an adjacent branch of the vessel. In response to such a challenge, attempts to simultaneously dilate both branches of the bifurcated vessel have been pursued. These attempts include deploying more than one balloon, more than one prosthesis, a bifurcated prosthesis, or some combination of the foregoing.

However, simultaneously deploying multiple and/or bifurcated balloons with or without endoluminal prostheses, hereinafter individually and collectively referred to as a bifurcated assembly, requires highly accurate placement of the assembly. Specifically, deploying a bifurcated assembly requires positioning a main body of the assembly within the trunk of the vessel adjacent the bifurcation, and then positioning the independent legs of the assembly into separately branching legs of the body lumen.

Tracking a bifurcated assembly to a treatment site also presents additional challenges to the more standard PTCA procedure. For example, a bifurcated catheter must be tracked to the site as a unitary device until it reaches the bifurcation. Once it reaches the bifurcated treatment site, it must be positioned within the separate branches of the vessel. Therefore, it must be a unitary device during tracking and be a bifurcated device for treatment.

In order to achieve the foregoing objectives, objectives, two guide wires are typically required, one for placement of the assembly into each branch of the bifurcated vessel. Devices known in the prior art fail to track and position a device requiring two guide wires in an expeditious fashion by failing to prevent the entanglement of the wires or other complications which would prevent proper placement of the assembly and/or a smooth withdrawal the catheter and of the guide wires.

Further, devices known in the prior art fail to provide a bifurcated assembly, the distal portion of which functions as a unitary device during tracking and as a bifurcated device for positioning and deployment.

In view of the foregoing, it is an object of this invention to provide improved catheters and methods for use with multiple guide wires for delivering balloon catheters and prostheses designed to treat stenoses at or near a bifurcation of a body lumen.

### BRIEF SUMMARY OF THE INVENTION

The present disclosure relates to bifurcated catheters which can be linked together such that they can be tracked to a bifurcated region of a body lumen over a single guide wire. A bifurcated catheter according to the present disclosure includes a first catheter branch having a first distal portion and a second catheter branch having a second distal portion. The first and second distal portions are linked together for delivery to the bifurcated region. Upon delivery to the bifurcated region, the first and second distal portions are released from each other such that the first and second catheter branches may be tracked into first and second vessel branches, respectively.

In an embodiment, the first and second distal portions may be linked together by a pocket disposed on the first distal portion. The pocket includes a proximal opening, a distal opening, a bore between the proximal and distal openings, and a slit from an outside surface of the pocket to the bore, the slit running from the proximal opening to the distal opening. The second distal portion is inserted into the pocket during delivery of the catheter to the bifurcated region, thereby coupling the first and second catheter portions to each other. The bifurcated catheter is tracked to the bifurcated region over a first guidewire disposed in the first catheter branch. Upon delivery of the catheter to the bifurcated region, a second guidewire is tracked through the second catheter branch and into a second branch vessel. As the catheter is advanced over both guidewires, the first and second catheter branches begin to separate, as the second distal portion is pulled through the slit in the pocket. The first and second catheter branches are then advanced into the first and second branch vessels.

Further features of the invention, its nature and various advantages, will be more apparent from the accompanying drawings and the following detailed description of the preferred embodiments.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a simplified, partial, elevational view of a bifurcated catheter in accordance with an embodiment of the present invention.

FIG. 2 illustrates a partial, elevational view of the distal portion of the catheter of FIG. 1.

FIG. 3 illustrates a partial, top view of the distal portion of the catheter of FIG. 1.

FIG. 3A illustrates a partial, top view of the distal portion of the catheter of FIG. 1 as the second distal portion is pulled through the slit of the pocket.

FIG. 3B is a sectional view of the pocket.

FIG. 4 illustrates the catheter of FIG. 1 in vivo, following the step of threading the catheter over a first guide wire.

FIG. 5 illustrates the catheter of FIG. 1 in vivo, when the catheter has been delivered to the bifurcation site.

FIG. 6 illustrates the catheter of FIG. 1 in vivo, as a second guidewire is advanced through the second catheter branch and into the second branch vessel.

FIG. 7 illustrates the catheter of FIG. 1 in vivo, as the second catheter branch is advanced over the second guidewire such that the second distal portion is pulled through the slit in the pocket.

FIG. 8 illustrates the catheter FIG. 1 in vivo, after the catheter branches have been advanced into the respective vessel branches.

FIG. 9 illustrates the catheter of FIG. 1 in vivo, subsequent to inflation of the balloon(s).

### DETAILED DESCRIPTION OF THE INVENTION

Specific embodiments of the present disclosure are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

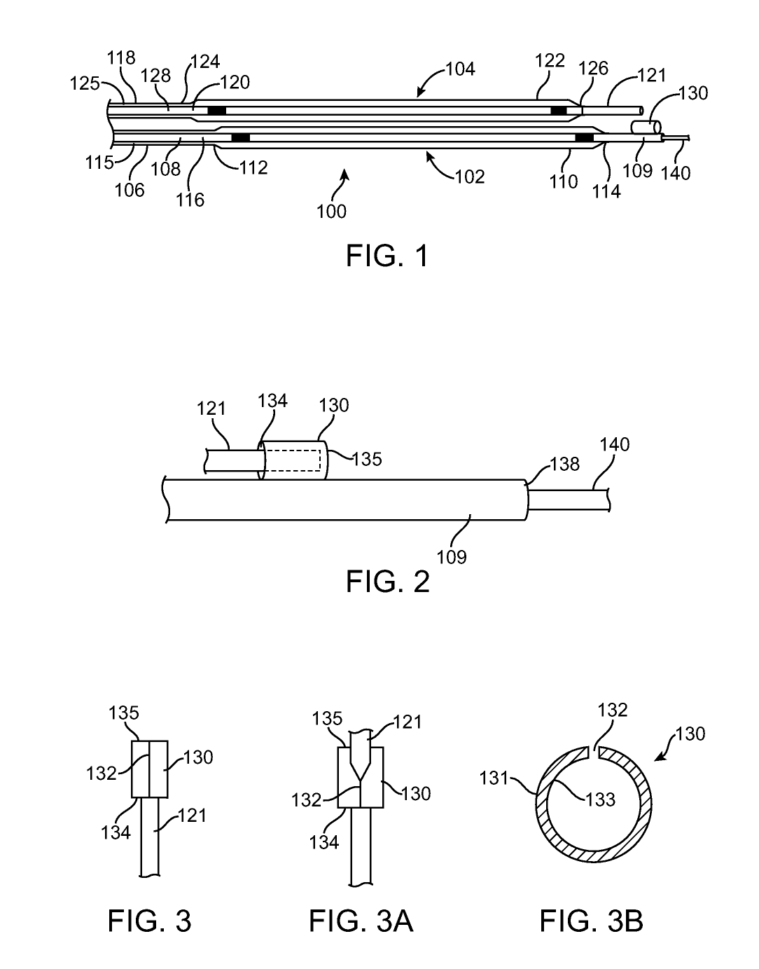

An illustrative embodiment of a catheter 100 constructed in accordance with this invention is shown in FIG. 1. The proximal portion of catheter 100 is toward the left in FIG. 1, and the distal portion is toward the right. Catheter 100 may comprise two separate tubular structures linked at particular points along their lengths, or it may consist of a single tubular structure with multiple lumens in its interior.

FIG. 1 depicts a catheter having two branches and two balloons, but more than two balloons may be utilized with the present invention. Alternatively, a bifurcated balloon, either alone or in combination with one or more standard balloons may be utilized.

Catheter 100 includes a first catheter branch 102 and a second catheter branch 104. First catheter branch 102 includes a first outer shaft 106, a first inner shaft 108, and a first balloon 110. A proximal end of first balloon 110 is mounted to a distal portion of first outer shaft 106 at a first proximal junction 112. A distal end of first balloon 110 is mounted to a distal portion 109 of first inner shaft 108 at a first distal junction 114. A first inflation lumen 115 extends between first outer shaft 106 and first inner shaft 108, and is in communication with an interior of first balloon 110. A first guidewire lumen 116 extends through first inner shaft 108.

Similarly, second catheter branch 104 includes a second outer shaft 118, a second inner shaft 120, and a second balloon 122. A proximal end of second balloon 122 is mounted to a distal portion of second outer shaft 118 at a second proximal junction 124. A distal end of second balloon 122 is mounted to a distal portion 121 of second inner shaft 120 at a second distal junction 126. A second inflation lumen 125 extends between second outer shaft 118 and second inner shaft 120, and is in communication with an interior of second balloon 122. A first guidewire lumen 128 extends through second inner shaft 120.

First and second inflation lumens 115, 125 can be conventional, and extend from a proximal portion of catheter 100 outside the patient, which is not pictured. First and second inflation lumens 115, 125 are in fluid communication with the interiors of first balloon 110 and second balloon 122. Thus, first and second inflation lumens 115, 125 are used to supply pressurized inflation fluid to first balloon 110 and second balloon 122 when it is desired to inflate the balloons. Inflation lumens 115,125 are also used to drain inflation fluid from first balloon 110 and second balloon 122 when it is desired to deflate the balloons.

Although first and second guidewire lumens 116, 128 are shown passing through the interior of first and second balloons 110, 122, they need not. For example, the lumens may be affixed to the exterior of the balloon, or the balloon may be formed with a plurality of folds through which the guide wire passes. Alternatively, the guide wire may pass through the folds of the balloon, as illustrated in U.S. Pat. No. 6,071,285 for a Rapid Exchange Folded Balloon Catheter and Stent Delivery System, the entirety of which is incorporated by reference herein. First and second guidewire lumens 116, 128 are distinct from first and second inflation lumens 115, 125 and are not in fluid communication with the interior of first and second balloons 110, 122. Further, first and second guidewire lumens 116, 128 can begin and terminate generally at any point along first and second catheter branches 102, 104, but preferably they extend distally of first and second balloons 110, 122, respectively. First and second guidewire lumens may extend proximally to the proximal end of catheter 100 (an over-the-wire design) or may terminate proximally of first and second balloons 110, 122 (a rapid-exchange type design). Preferably, first catheter branch 102 is a rapid-exchange type design and second catheter branch 104 is an over-the-wire type design.

First catheter branch 102 further includes pocket 130 mounted to distal portion 109, as shown in FIG. 2. Pocket 130 may be adhesively or mechanically bonded to distal portion 109. Pocket 130 is mounted to distal portion 109 such that pocket 130 is directed towards second distal portion 121 of second inner shaft 120. Pocket 130 may be an open cylinder with a proximal opening 134 and a distal opening 135, as shown in FIGS. 2, 3, and 3A. Pocket 130 further includes a slit 132 that extends from an outer surface 131 of pocket 130 to an inner surface 133 of pocket 133, as shown in FIG. 3B. Pocket 130 is sized and shaped to snugly receive distal portion 121 of second inner shaft 120. By inserting distal portion 121 into pocket 130, first catheter branch 102 and second catheter branch 104 are coupled together for delivery to the site of a lesion. Once at the lesion site, distal portion 121 is pulled through slit 132 of pocket 130, as shown in FIG. 3A and explained in more detail below, thereby freeing second catheter branch 104 from first catheter branch 102.

Pocket 130 may be made from the same materials as would be commonly used for catheters, as would be recognized by one of ordinary skill in the art. Further, by describing pocket 130 as being coupled to distal portion 109 of first inner shaft 108, one of ordinary skill in the art would understand that pocket 130 may be made unitary with distal portion 109.

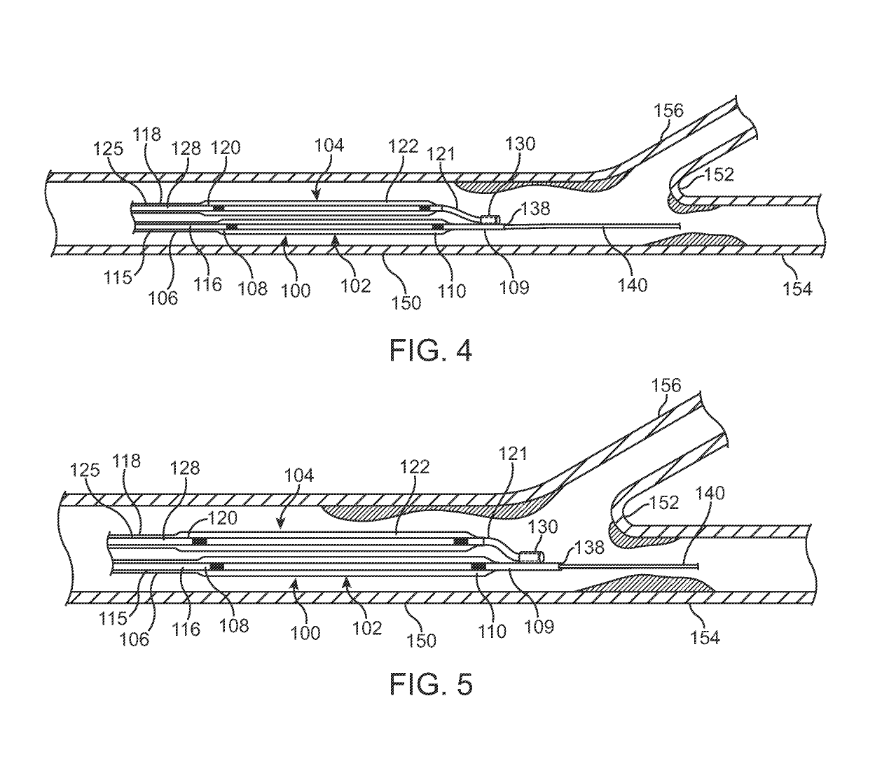

With reference to FIGS. 4-9, an exemplary manner of practicing the invention will now be discussed. First catheter branch 102 is threaded over a first guidewire 140 which is already in place in the body lumen. More specifically, a proximal end of first guidewire 140 is threaded into a distal opening 138 of distal portion 109 of first inner shaft 108, and through first guidewire lumen 116. First catheter branch 102 is threaded over first guidewire 140 while distal portion 121 of second inner shaft 120 is inserted into pocket 130, thereby coupling the distal portions of first and second catheter branches 102, 104 together, as shown in FIG. 4.

Catheter 100 is thus threaded over first guide wire 140 and tracked to a position at or near a bifurcation 152 of a vessel 150, as depicted in FIG. 5. A second guidewire 142 may be pre-installed through second guidewire lumen 128 such that second guidewire 142 is advanced with catheter 100 as catheter 100 is advanced to the bifurcation site 152. Alternatively, second guide wire 142 may be inserted in second guidewire lumen 128 after catheter 100 has reached the bifurcation site 152.

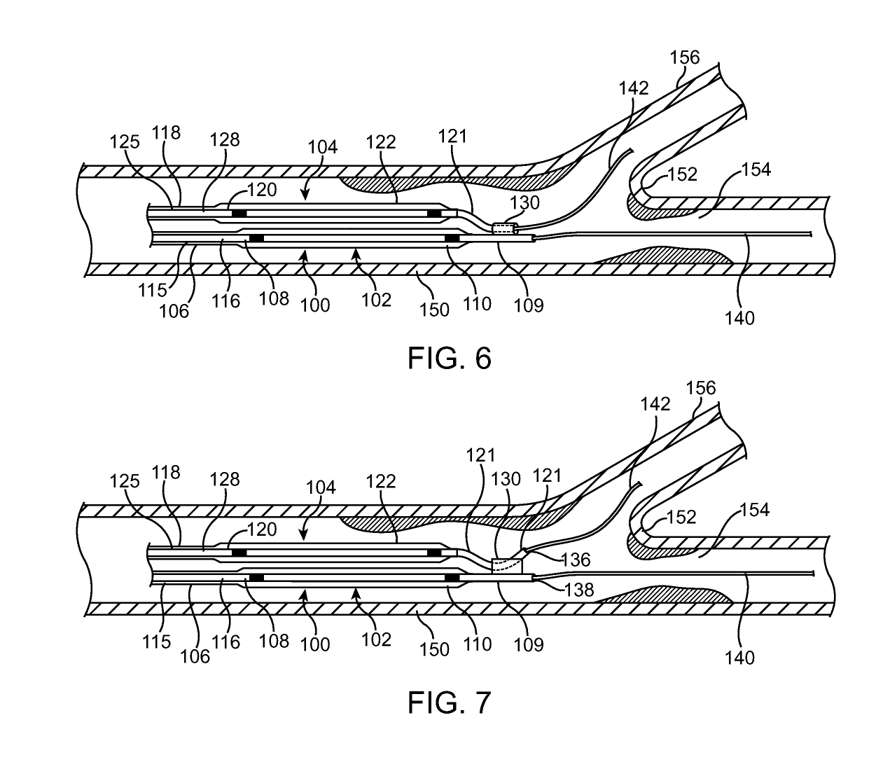

Once catheter 100 is near bifurcation 152, second guidewire 142 is advanced through a distal opening 136 in distal portion 121 of second inner shaft 120 and into a second branch vessel 156, as illustrated in FIG. 6.

Catheter 100 is then advanced over guidewires 140, 142. As catheter 100 approaches bifurcation 152, first catheter branch 102, which is tracking over first guidewire 142, tracks towards first branch vessel 154. Meanwhile, second catheter branch 104, which is tracking over second guidewire 442, tracks towards second branch vessel 156. The divergent paths of first catheter branch 102 and second catheter branch 104 causes distal portion 121 of second inner shaft 120 to exert pressure against slit 132 of pocket 130, thereby opening slit 130, as shown in FIG. 7. As first and second catheter branches continue to move apart from one another, distal portion 121 withdraws completely from pocket 130, thereby freeing first and second catheter branches 102, 104 from each other.

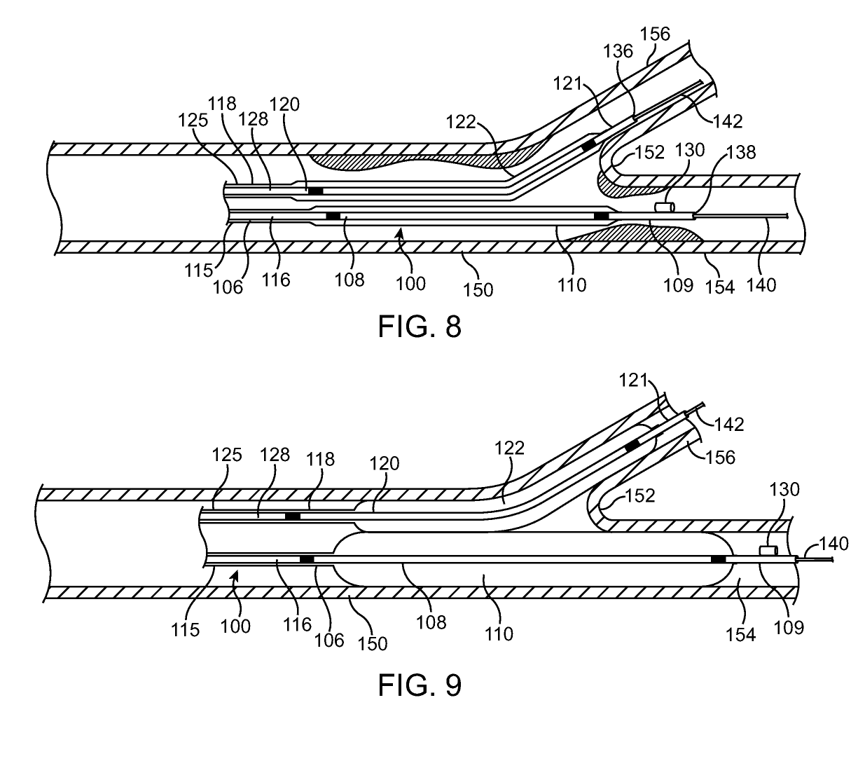

With distal portion 121 of second inner shaft released from pocket 130, first and second branches 102, 104 can then be positioned independently of one another such that first and second balloons 110, 122 may be positioned independently of each other. First and second catheter branches 102, 104 are advanced into first and second branch vessels 154, 156, respectively, as illustrated in FIG. 8.

Once the entire assembly is properly positioned, pressurized fluid is supplied to first and second balloons 110, 122 through first and second inflation lumens 115,125, as shown in FIG. 9. After first balloon 110 and second balloon 122 have been inflated as described above, first balloon 110 and second balloon 122 are deflated by draining the inflation fluid via first and second inflation lumens 115, 125. This allows the balloons to collapse in preparation for withdrawal of the assembly from vessel 150.

As would be understood by those of ordinary skill in the art, a bifurcated stent may be mounted on first and second balloons 110, 122 of catheter 100, as shown in FIGS. 2-2F of U.S. Pat. No. 6,129,738, the entirety of which is incorporated by reference herein. As noted in the '738 patent, a single bifurcated stent or multiple stents, in place of or in combination with a bifurcated stent, may be deployed utilizing a bifurcated catheter of the present invention.

The various components of the catheters of this invention can be made of the same materials that are conventionally used for generally corresponding components of known catheters. Thus, for example, the various lumens can be made of materials such as polyethylene, polyethylene terephthalate, polyurethanes, polyesters, polyamides and copolymers thereof.

As another example, at least part of the outer or inner shafts may be stainless steel, polyimide or the like. A polyimide hyptotube or similar material may encase the proximal shaft of the catheter. A sufficiently rigid material may prevent the twisting of the catheter and potential distortion of the lumens and guide wires within the catheter in the event a torque is applied to the catheter during positioning of the device.

The material of the balloons may be polyethylene, polyethylene terephthalate, nylon, polyamides, latex rubber, or other polymer. Guide wires can also be of any conventional construction and material, including solid or braided stainless steel. Hence, the term “wire” is used for these elements only as a matter of convenience, and that the material may not necessarily be wire.

The dimensions (e.g., the lengths, diameters, thicknesses, etc.) of various components of the catheters of this invention may be similar to the dimensions that are conventionally used for generally corresponding components of known catheters.

It would be understood by those of ordinary skill in the art that while the embodiments of the present invention discussed above are described with respect to a dual-lumen catheter including an outer shaft and an inner shaft, several different types of catheters known in the art could be used, for example, rapid exchange type catheters.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. An apparatus for treating a bifurcated region of a body lumen, comprising:
a catheter having a first catheter branch and a second catheter branch, wherein the first catheter branch includes a first distal portion and the second catheter branch includes a second distal portion;
a pocket disposed on the first portion, the pocket including a proximal opening, a distal opening, and a slit extending from an exterior surface to an interior surface of the pocket, wherein the pocket is directed towards the second catheter branch and wherein the pocket is sized and shaped to receive the second distal portion through the proximal opening.

2. The apparatus of claim 1, further comprising at least one balloon disposed on the first and second catheter branches, wherein the first and second distal portions are disposed distal of the balloon.

3. The apparatus of claim 2, wherein the first catheter branch includes a first inflation lumen in communication with an interior of the balloon and the second catheter branch includes a second inflation lumen in communication with the balloon.

4. The apparatus of claim 1, further comprising a first balloon disposed on the first catheter branch and a second balloon disposed on the second catheter branch.

5. The apparatus of claim 4, wherein the first catheter branch includes a first inflation lumen in communication with an interior of the first balloon and the second catheter branch includes a second inflation lumen in communication with the second balloon.

6. The apparatus of claim 1, wherein the first catheter branch includes a first guide wire lumen and the second catheter branch includes a second guide wire lumen.

7. The apparatus of claim 1, wherein the second distal portion is disposed within the pocket during delivery to the bifurcated region, and the second distal portion is pulled through the slit to dislodge the second distal portion from the pocket after delivery to the bifurcated region.

8. The apparatus of claim 1, wherein the pocket is generally cylindrical.

9. A method for treating a bifurcated region of a body lumen, comprising the steps of:
providing a catheter having a first catheter branch, a second catheter branch, and at least one balloon disposed on the first and second catheter branches, wherein the first catheter branch includes a first distal portion and the second catheter branch includes a second distal portion, wherein the first distal portion includes a pocket coupled thereto, the pocket including a proximal opening, a distal opening, and a slit extending from an exterior surface to an interior surface of the pocket;
inserting the second distal portion into the proximal opening of the pocket;
inserting a first guidewire into the body lumen and advancing the first guidewire to the bifurcated region;
inserting a proximal end of the first guidewire into a first distal opening of the first distal portion;
advancing the catheter over the first guidewire to the bifurcated region;
advancing a second guidewire through a guidewire lumen in the second catheter branch, out of a distal opening in the second distal portion, out of the distal opening of the pocket, and into a first branch of the body lumen;
advancing the first guidewire into a second branch of the body lumen;
advancing the bifurcated catheter over the first and second guidewires such that the first catheter branch advances into the second branch of the body lumen and the second catheter branch advances into the first branch of the body lumen, wherein the second distal portion is pulled through the slit of the pocket due to a force of the first and second catheter branches pulling apart due to the advancement into the second and first branch vessels, respectively; and
inflating the at least one balloon.

10. The method of claim 9, wherein the first catheter branch includes a first inflation lumen in communication with an interior of the at least one balloon and the second catheter branch includes a second inflation lumen in communication with the at least one balloon.

11. The method of claim 9, wherein the at least one balloon comprises a first balloon disposed on the first catheter branch and a second balloon disposed on the second catheter branch.

12. The method of claim 11, wherein the first catheter branch includes a first inflation lumen in communication with an interior of the first balloon and the second catheter branch includes a second inflation lumen in communication with the second balloon.
